(19) United States
(12) Patent Application Publication
(10) Pub. No.: US 2023/0124250 A1
(43) Pub. Date: Apr. 20, 2023

(54) SANITARY FINGER COVER FOR USE WITH A FINGER PROBE

(71) Applicant: Charisse Alvarez, Plantation, FL (US)

(72) Inventor: Charisse Alvarez, Plantation, FL (US)

(21) Appl. No.: 17/502,754

(22) Filed: Oct. 15, 2021

(51) Int. Cl. A61B 5/00; A61B 42/20; A61B 5/145

(52) U.S. Cl. CPC A61B 5/6826; A61B 42/20; A61B 5/14542

(57) ABSTRACT

A sanitary finger cover that includes a cover body of an elastic material, an outer surface, a first end defining an enclosed cover opening, an inner surface defining an enclosed cover channel, a second end, and a cover length of at least 1″ and separating the first and second ends. The cover body has an enclosed sidewall spanning the cover length and configured to at least partially enclose the finger of the patient and cover the tip of the finger of the patient enclosed and second end adapted to cover the tip of the finger. Additionally, the cover assembly includes a finger probe operably configured to measure user biometrics through, and at least partially superimposed over and directly coupled to, the outer surface of the cover body, thereby enabling the cover body to provide a barrier between the finger of the patient and the finger probe.

### FIELD OF THE INVENTION

The present invention relates generally to covers for a user's finger and configured for use with a finger probe.

### BACKGROUND OF THE INVENTION

In the medical field, hygiene is important for minimizing the spread of disease. Common medical hygiene practices include sterilization of instruments and surfaces (including biological surfaces and the surfaces with which they contact), the use of protective barriers (e.g., masks, gowns, caps, eyewear and gloves), safe disposal of medical waste, disinfection of reusables, and handwashing.

Many patients require a finger probe, which is often used for pulse oximetry testing and monitoring blood oxygen saturation. Current procedures involve the finger probe being placed over the patient's finger, whereon the finger probe remains throughout the monitoring process. The finger probe is then removed from the finger and may be reused on other patients. In the medical settings, the finger probe is not cleaned by healthcare professionals resulting in the potential for contamination of the finger probe and the spread of infection between patients

Therefore, a need exists to overcome the problems with the prior art as discussed above.

### SUMMARY OF THE INVENTION

The invention provides a sanitary finger cover for use with a finger probe that overcomes the hereinafore-mentioned disadvantages of the heretofore-known devices and methods of this general type and that enables user's, e.g., patients, to protect themselves from contamination.

With the foregoing and other objects in view, there is provided, in accordance with the invention, a sanitary finger cover that includes a cover body of an elastic material, with an outer surface, with a first end defining an enclosed cover opening sized to receive a finger of a patient, with an inner surface defining an enclosed cover channel sized to receive the finger of the patient, with a second end opposing the first end of the cover body, and with a cover length of at least 1″ and separating the first and second ends of the cover body, wherein the cover body has an enclosed sidewall spanning the cover length and is configured to at least partially enclose the finger of the patient and cover the tip of the finger of the patient and the second end adapted to cover the tip of the finger. A finger probe is utilized that is operably configured to measure user biometrics through, and at least partially superimposed over and directly coupled to, the outer surface of the cover body, thereby enabling the cover body to provide a barrier between the finger of the patient and the finger probe.

In accordance with another feature, an embodiment of the present invention includes an elastic band defining the first end and enclosing the cover opening.

In accordance with a further feature of the present invention, the enclosed sidewall of the cover body of a thin-walled material and of a thickness less than or equal to 1/32″. Additionally, the enclosed cover opening is approximately 0.5-1″. Further, the thickness of the enclosed sidewall may uniformly and continuously spans at least 90% of the cover length.

In accordance with yet another feature of the present invention, the finger probe is permanently affixed to the outer surface cover body.

In accordance with an exemplary feature of the present invention, the finger probe is operably configured to measure blood oxygen saturation of the patient.

Also in accordance with the present invention and in combination with a finger probe operably configured to measure user biometrics through a finger of a patient, an improvement is disclosed that includes a sanitary finger cover having a cover body of an elastic material, with an outer surface, with a first end defining an enclosed cover opening sized to receive a finger of a patient, with an inner surface defining an enclosed cover channel sized to receive the finger of the patient, with a second end opposing the first end of the cover body, and with a cover length of at least 1″ and separating the first and second ends of the cover body, wherein the cover body has an enclosed sidewall spanning the cover length and configured to at least partially enclose the finger of the patient and cover the tip of the finger of the patient enclosed and with second end adapted to cover the tip of the finger and the finger probe is at least partially superimposed over and directly coupled to, the outer surface of the cover body, thereby enabling the cover body to provide a barrier between the finger of the patient and the finger probe.

Although the invention is illustrated and described herein as embodied in a sanitary finger cover for use with a finger probe, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward. The figures of the drawings are not drawn to scale.

Before the present invention is disclosed and described, it is to be understood that the terminology used herein is for the purpose of describing particular embodiments only and is not intended to be limiting. The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “providing” is defined herein in its broadest sense, e.g., bringing/coming into physical existence, making available, and/or supplying to someone or something, in whole or in multiple parts at once or over a period of time. Also, for purposes of description herein, the terms “upper”, “lower”, “left,” “rear,” “right,” “front,” “vertical,” “horizontal,” and derivatives thereof relate to the invention as oriented in the figures and is not to be construed as limiting any feature to be a particular orientation, as said orientation may be changed based on the user's perspective of the device. Furthermore, there is no intention to be bound by any expressed or implied theory presented in the preceding technical field, background, brief summary or the following detailed description.

As used herein, the terms “about” or “approximately” apply to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. In this document, the term “longitudinal” should be understood to mean in a direction corresponding to an elongated direction of the finger cover, spanning from the first end of the finger cover to the second end of the finger cover.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

FIG. 1 is an elevational front view of a sanitary finger cover for use with a finger probe in accordance with one embodiment of the present invention;

FIG. 2 is an elevational front view of the sanitary finger cover of FIG. 1 in a removed configuration from a patient's finger in accordance with one embodiment of the present invention;

FIG. 3 is an elevational front view of the sanitary finger cover of FIG. 1 in an operational configuration on the patient's finger in accordance with one embodiment of the present invention;

FIG. 4 is an elevational front view of the sanitary finger cover of FIG. 1 in an operational configuration on the patient's finger with a finger probe directly coupled thereto;

FIG. 5 is a fragmentary cross-sectional view of a sanitary finger cover in accordance with one embodiment of the present invention; and

FIG. 6 is a process flow diagram depicting an exemplary method of coupling a sanitary finger cover to a patient's finger and a finger probe in accordance with one embodiment of the present invention.

### DETAILED DESCRIPTION

While the specification concludes with claims defining the features of the invention that are regarded as novel, it is believed that the invention will be better understood from a consideration of the following description in conjunction with the drawing figures, in which like reference numerals are carried forward. It is to be understood that the disclosed embodiments are merely exemplary of the invention, which can be embodied in various forms.

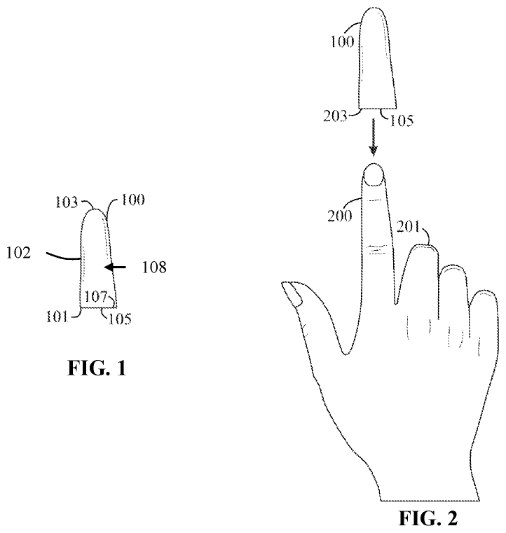

The present invention provides a novel and efficient sanitary finger cover for use with a finger probe. Referring now to FIGS. 1-4, one embodiment of the present invention is shown in various views. The figures show several advantageous features of the present invention, but, as will be described below, the invention can be provided in several shapes, sizes, combinations of features and components, and varying numbers and functions of the components. Specifically, FIG. 1 illustrates a sanitary finger cover 100 having a cover body 102 with a first end 101 and a second end 103 opposing the first end 101 of the cover body 102. The first end 101 defines an opening 105 that is preferably enclosed and is sized and shaped to receive a finger 200 of a patient 201 prior to positioning the finger probe over the finger cover 100.

The second end 103 of the cover body 102 is operably configured to cover the tip of the patient's finger. In some embodiments, the first end 101 may include an elastic material 107 positioned around the perimeter of the opening 105. The elastic material 107 is operably configured to retain the finger cover 100 in a suitable position on the finger of the patient to prevent contact between the patient's skin and the finger probe.

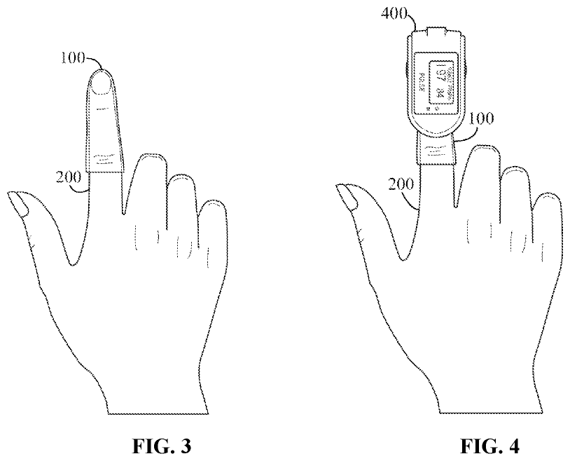

FIG. 2 illustrates the finger cover 100 disengaged from the finger 200 of the patient 201, wherein FIGS. 3-4 depict the finger cover 100 engaged or removably coupled to the user's finger 201 and a finger probe 400 removably coupled to the outer surface of the finger cover 100. The finger cover 100 is dimensioned to at least partially contain a length of the finger 200, such that the finger 200 does not contact the finger probe 400 to promote sanitary practices in the healthcare field. The perimeter 203 of the opening 105 is dimensioned to be retained around the circumference of the finger 200 to retain the finger cover 100 in a suitable position during use and permit the finger probe 400 to measure biometrics, e.g., blood oxygen saturation.

Specifically, FIG. 3 illustrates the finger cover 100 retained on the finger 200 of the patient 201. During use, the finger cover 100 is positioned over the finger 200 such that the finger cover 100 at least partially covering the finger of the patient. In some embodiments, the finger cover 100, specifically the cover body 102, is constructed of a flexible and elastic material, e.g., silicone rubber. to allow the finger cover 100 to be used on patients with various hand and finger sizes. The finger cover 100 is preferably constructed as a single-use and disposable item.

FIG. 4 illustrates the finger probe 400 positioned over the finger cover 100 to allow the finger probe 400 to measure and monitor biometric information when engaged with the finger 200. The cover body 102 is of a thin thickness to allow for biometric measurements to be received from the finger 200 while forming a barrier between the skin and the finger probe.

In some embodiments, the finger cover 100 extends along the length of the finger 200 and has a length longer than the length of the finger probe 400 to ensure the finger 200 does not contact the finger probe 400. For example, some exemplary dimensions of the cover body 102 include a length of approximately 2 inches, a diameter of approximately 0.5 inches, and a circumference of the first end 101 that is approximately 1.5 inches. Further, the finger probe 400 may be permanently affixed to the outer surface 108 cover body 102 using, for example, adhesive, welding, etc.

The cover body 102 can also be seen having an outer surface 108, a first end 101 defining an enclosed cover opening 105 sized to receive a finger of a patient, an inner surface 502 defining an enclosed cover channel 504 sized to receive the finger of the patient, and a second end 103 opposing the first end 101 of the cover body 102. The cover length may beneficially be at least 1″ and separates the first and second ends 101, 103 of the cover body 102. In one embodiment, the cover channel 504 may extend approximately the entire cover length and may be of a shape similar to the user's finger. The outer surface shape may be of a similar shape or may be of an oblong or other shape. The cover body 102 can also be seen having an enclosed sidewall 500 spanning the cover length and is configured to at least partially enclose the finger of the patient and cover the tip of the finger of the patient enclosed and second end adapted to cover the tip of the finger.

The finger probe 400 is operably configured to measure user biometrics through, and at least partially superimposed over and directly coupled to, the outer surface 108 of the cover body 102, thereby enabling the cover body 102 to provide a barrier between the finger of the patient and the finger probe 400. The elastic band 107, which may be formed by overlaying layers of material or having a separate band of material, defines the first end 101 and encloses the cover opening 105. In one preferred embodiment, the finger probe 400 is operably configured to measure blood oxygen saturation of the patient. The finger probe 400 may be manipulated to expand and contract over all or a portion of the user's finger where the cover body 102 is directly coupled.

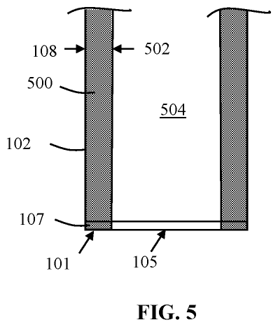

As used herein, the term “wall” is intended broadly to encompass continuous structures, as well as, separate structures that are coupled together so as to form a substantially continuous external surface. As seen in FIG. 5, the enclosed sidewall 500 of the cover body 102 if of a thin-walled material and has a thickness less than or equal to approximately 1/32″. The thickness of the enclosed sidewall 500 may also uniformly and continuously span at least 90% of the cover length, but preferably spans in a uniform width from the elastic band 107 to the second end 103. Other exemplary but preferred dimensions include the enclosed cover opening 105 being approximately 0.5-1″, wherein the cover length is approximately 1.5-2 77″, the outer diameter separating two opposing sides of the outer surface 108 is approximately 0.5-1″, and the inner diameter separating two opposing sides of the inner surface 502 is approximately 0.5-1″. As the sanitary cover body 102 may be of an elastic material, such as food-grade silicone, said dimensions may dynamically vary. Said dimensions are important because a sufficient length of material is needed to ensure non-contact between the patient's finger and the probe 400. The flexible and elastic cover body 102 may, in some embodiments, also be breathable.

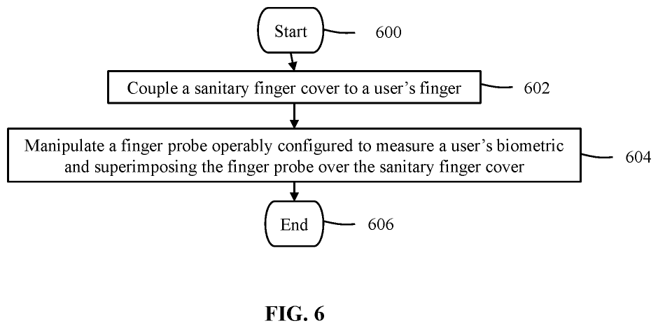

In one exemplary method of using the cover 100, as depicted in FIG. 6, the process begins as step 600 and immediately proceeds to step 602 coupling the cover body 102 to a user's finger using the above-described structure of the cover body 102. The user may, for example, elastically deform the cover to open the cover opening 105 a diameter sufficient to receive a user's finger. Then, the user will release the cover body 102 to retain the cover body 102 on the user's patient. Then step 604 may include manipulating a finger probe 400 that is operably configured to measure a user's biometric and superimposing the finger probe 400 over the sanitary finger cover 100, thereby shielding the user's finger from contamination. The process may terminate at step 606.

As such, the process flow chart in FIG. 5 should be read in light of the above-described disclosure. Although FIG. 5 shows a specific order of executing the process steps, the order of executing the steps may be changed relative to the order shown in certain embodiments. Also, two or more blocks shown in succession may be executed concurrently or with partial concurrence in some embodiments. Certain steps may also be omitted in FIG. 5 for the sake of brevity. In some embodiments, some or all of the process steps included in FIG. 5 can be combined into a single process.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the above described features.

What is claimed is:

1. A sanitary finger cover comprising:
a cover body of an elastic material, with an outer surface, with a first end defining an enclosed cover opening sized to receive a finger of a patient, with an inner surface defining an enclosed cover channel sized to receive the finger of the patient, with a second end opposing the first end of the cover body, and with a cover length of at least 1″ and separating the first and second ends of the cover body, the cover body having an enclosed sidewall spanning the cover length and configured to at least partially enclose the finger of the patient and cover the tip of the finger of the patient and with the second end adapted to cover the tip of the finger; and
a finger probe operably configured to measure user biometrics through, and at least partially superimposed over and directly coupled to, the outer surface of the cover body, thereby enabling the cover body to provide a barrier between the finger of the patient and the finger probe.

2. The sanitary finger cover according to claim 1, further comprising:
an elastic band defining the first end and enclosing the cover opening.

3. The sanitary finger cover according to claim 1, wherein:
the enclosed sidewall of the cover body of a thin-walled material and a thickness less than or equal to 1/32″.

4. The sanitary finger cover according to claim 3, wherein:
the enclosed cover opening is approximately 0.5-1″.

5. The sanitary finger cover according to claim 3, wherein:
the thickness of the enclosed sidewall uniformly and continuously spans at least 90% of the cover length.

6. The sanitary finger cover according to claim 1, wherein:
the finger probe is permanently affixed to the outer surface cover body.

7. The sanitary finger cover according to claim 1, wherein:
the finger probe is operably configured to measure blood oxygen saturation of the patient.

8. In combination with a finger probe operably configured to measure user biometrics through a finger of a patient, the improvement comprising:
a sanitary finger cover having a cover body of an elastic material, with an outer surface, with a first end defining an enclosed cover opening sized to receive a finger of a patient, with an inner surface defining an enclosed cover channel sized to receive the finger of the patient, with a second end opposing the first end of the cover body, and with a cover length of at least 1″ and separating the first and second ends of the cover body, the cover body having an enclosed sidewall spanning the cover length and configured to at least partially enclose the finger of the patient and cover the tip of the finger of the patient and with the second end adapted to cover the tip of the finger and the finger probe is at least partially superimposed over and directly coupled to, the outer surface of the cover body, thereby enabling the cover body to provide a barrier between the finger of the patient and the finger probe.

9. The improvement according to claim 8, further comprising:
an elastic band defining the first end and enclosing the cover opening.

10. The improvement according to claim 8, wherein:
the enclosed sidewall of the cover body if of a thin-walled material and a thickness less than or equal to 1/32″.

11. The improvement according to claim 10, wherein:
the enclosed cover opening is approximately 0.5-1″.

12. The improvement according to claim 10, wherein:
the thickness of the enclosed sidewall uniformly and continuously spans at least 90% of the cover length.

13. The improvement according to claim 8, wherein:
the finger probe is permanently affixed to the outer surface cover body.

14. The improvement according to claim 8, wherein:
the finger probe is operably configured to measure blood oxygen saturation of the patient.
